(19) United States
(12) Patent Application Publication
(10) Pub. No.: US 2013/0004602 A1
(43) Pub. Date: Jan. 3, 2013

(54) LOTION COMPOSITION AND FIBROUS STRUCTURE COMPRISING SAME

(76) Inventors: Michael Raymond Mootz, Cincinnati, OH (US); Angela Marie Leimbach, Hamilton, OH (US)

(21) Appl. No.: 13/537,339

(22) Filed: Jun. 29, 2012

Related U.S. Application Data
(60) Provisional application No. 61/503,678, filed on Jul. 1, 2011.

(51) Int. Cl. A61K 47/44; A61K 36/886; A61K 36/18; C10G 73/00

(52) U.S. Cl. 424/744; 208/24; 424/776; 514/789

(57) ABSTRACT

A lotion composition containing a microcrystalline wax, a fibrous structure containing such lotion composition and a method for making such fibrous structure and/or lotion composition are provided.

### CROSS REFERENCE TO RELATED APPLICATION

This application claims the benefit of U.S. Provisional Application No. 61/503,678 filed Jul. 1, 2011.

### FIELD OF THE INVENTION

This invention relates to a lotion composition, and more particularly to a lotion composition comprising a microcrystalline wax, a fibrous structure comprising such lotion composition and a method for making such fibrous structure and/or lotion composition.

### BACKGROUND OF THE INVENTION

Lotion compositions and fibrous structures, such as sanitary tissue products, for example facial tissue and bath tissue, comprising lotion compositions are known in the art. All of these lotion containing sanitary tissue products share a common need, specifically to be soft to the touch. Softness is a complex tactile impression elicited by a product when it is stroked against the skin. The purpose of being soft is so that these products can be used to cleanse the skin without being irritating. Effectively cleansing the skin is a persistent personal hygiene problem for many people. Objectionable discharges of urine, menses, and fecal matter from the perineal area or otorhinolaryngogical mucus discharges do not always occur at a time convenient for one to perform a thorough cleansing, as with soap and copious amounts of water for example. As a substitute for thorough cleansing, a wide variety of tissue and toweling products are offered to aid in the task of removing from the skin and retaining the before mentioned discharges for disposal in a sanitary fashion. Not surprisingly, the use of these products does not approach the level of cleanliness that can be achieved by the more thorough cleansing methods, and producers of tissue and toweling products are constantly striving to make their products compete more favorably with thorough cleansing methods.

Accordingly, making soft tissue and toweling products which promote comfortable cleaning without performance impairing sacrifices has long been the goal of the engineers and scientists who are devoted to research into improving tissue paper. There have been numerous attempts to reduce the abrasive effect, i.e., improve the softness of tissue products.

One area that has been exploited in this regard has been to select and modify cellulose fiber morphologies and engineer paper structures to take optimum advantages of the various available morphologies. Applicable art in this area include in U.S. Pat. Nos. 5,228,954; 5,405,499; 4,874,465; and 4,300,981. Another area which has received a considerable amount of attention is the addition of chemical softening agents (also referred to herein as “chemical softeners”) to tissue and toweling products.

In addition to attempts described above, formulators have also attempted to improve lotion compositions such that they provide improved softness to sanitary tissue products. A lotion composition comprising a microcrystalline wax and a sanitary tissue product comprising such lotion composition is even known in the art. Even in light of these attempts by formulators to produce less abrasive, softer sanitary tissue products by improving lotion compositions, consumers still have a need for sanitary tissue products that provide improved softness.

Accordingly, there is a need for a lotion composition that when applied to a surface of a fibrous structure, for example a sanitary tissue product, provides improved softness over known lotion compositions and/or fibrous structures comprising such lotion compositions.

### SUMMARY OF THE INVENTION

The present invention fulfills the need described above by providing a lotion composition comprising a microcrystalline wax and fibrous structures, for example sanitary tissue products comprising such lotion composition, wherein the lotion composition exhibits novel properties compared to known lotion compositions.

In one example of the present invention, a lotion composition comprising a microcrystalline wax, wherein the lotion composition exhibits a penetration hardness of 65 or less and/or 60 or less and/or 55 or less and/or 50 or less and/or 48 or less and/or 46 or less as measured according to the Penetration Hardness Test Method described herein is provided.

In another example of the present invention, a lotion composition comprising a microcrystalline wax, wherein the lotion composition exhibits a viscosity at 65° C. of 50 or less and/or 45 or less and/or 40 or less and/or 35 or less and/or greater than 5 and/or greater than 10 and/or greater than 15 and/or greater than 20 l/seconds (l/s) is provided.

In yet another example of the present invention, a lotion composition comprising a microcrystalline wax, wherein the lotion composition exhibits a Kinectic Coefficient of Friction of less than 1 and/or less than 0.8 and/or less than 0.7 and/or greater than 0.1 and/or greater than 0.3 and/or greater than 0.5 as measured according to the Kinetic Coefficient of Friction Test Method described herein is provided.

In still another example of the present invention, a fibrous structure, for example a sanitary tissue product, such as a facial tissue or bath tissue, comprising a lotion composition according to the present invention is provided.

In even another example of the present invention, an article of manufacture comprising a carton comprising a plurality of fibrous structures, for example a plurality of sanitary tissue products, such as facial tissues or bath tissue, according to the present invention is provided.

Accordingly, the present invention provides a lotion composition comprising a microcrystalline wax the exhibits novel properties and a fibrous structure, such as a sanitary tissue product, comprising such lotion composition.

### DETAILED DESCRIPTION OF THE INVENTION

### Definitions

“Microcrystalline wax” as used herein means a refined mixture of solid, saturated aliphatic hydrocarbons and is produced by de-oiling certain fractions from the petroleum refining process. Microcrystalline waxes differ from refined paraffin wax in that the molecular structure is more branched and the hydrocarbon chains are longer (higher molecular weight). As a result the crystal structure of microcrystalline wax is much finer than paraffin wax, and this directly impacts many of the physical properties. Microcrystalline waxes are tougher, more flexible and generally higher in melting point than paraffin wax. The fine crystal structure also enables microcrystalline wax to bind solvents or oil, and thus prevent the sweating-out of compositions. There are three categories of microcrystalline wax based on its melting point. A first category of microcrystalline wax exhibits a melting point of about 54 to about 76° C. A second category of microcrystalline wax exhibits a melting point of about 76 to about 85° C. A third category of microcrystalline wax exhibits a melting point of about 85 to about 95° C.

“Fiber” as used herein means an elongate particulate having an apparent length greatly exceeding its apparent diameter, i.e. a length to diameter ratio of at least about 10. Fibers having a non-circular cross-section are common; the “diameter” in this case may be considered to be the diameter of a circle having cross-sectional area equal to the cross-sectional area of the fiber. More specifically, as used herein, “fiber” refers to papermaking fibers. The present invention contemplates the use of a variety of papermaking fibers, such as, for example, natural fibers or synthetic fibers, or any other suitable fibers, and any combination thereof.

Natural papermaking fibers useful in the present invention include animal fibers, mineral fibers, plant fibers and mixtures thereof. Animal fibers may, for example, be selected from the group consisting of: wool, silk and mixtures thereof. Plant fibers may, for example, be derived from a plant selected from the group consisting of: wood, cotton, cotton linters, flax, sisal, abaca, hemp, hesperaloe, jute, bamboo, bagasse, kudzu, corn, sorghum, gourd, agave, loofah and mixtures thereof.

Wood fibers; often referred to as wood pulps include chemical pulps, such as kraft (sulfate) and sulfite pulps, as well as mechanical and semi-chemical pulps including, for example, groundwood, thermomechanical pulp, chemi-mechanical pulp (CMP), chemi-thermomechanical pulp (CTMP), neutral semi-chemical sulfite pulp (NSCS). Chemical pulps, however, may be preferred since they impart a superior tactile sense of softness to tissue sheets made therefrom. Pulps derived from both deciduous trees (hereinafter, also referred to as “hardwood”) and coniferous trees (hereinafter, also referred to as “softwood”) may be utilized. The hardwood and softwood fibers can be blended, or alternatively, can be deposited in layers to provide a stratified and/or layered fibrous structure. U.S. Pat. No. 4,300,981 and U.S. Pat. No. 3,994,771 are incorporated herein by reference for the purpose of disclosing layering of hardwood and softwood fibers. Also applicable to the present invention are fibers derived from recycled paper, which may contain any or all of the above categories as well as other non-fibrous materials such as fillers and adhesives used to facilitate the original papermaking.

The wood pulp fibers may be short (typical of hardwood fibers) or long (typical of softwood fibers). Nonlimiting examples of short fibers include fibers derived from a fiber source selected from the group consisting of Acacia, Eucalyptus, Maple, Oak, Aspen, Birch, Cottonwood, Alder, Ash, Cherry, Elm, Hickory, Poplar, Gum, Walnut, Locust, Sycamore, Beech, Catalpa, Sassafras, Gmelina, Albizia, Anthocephalus, and Magnolia. Nonlimiting examples of long fibers include fibers derived from Pine, Spruce, Fir, Tamarack, Hemlock, Cypress, and Cedar. Softwood fibers derived from the kraft process and originating from more-northern climates may be preferred. These are often referred to as northern softwood kraft (NSK) pulps.

Synthetic fibers may be selected from the group consisting of: wet spun fibers, dry spun fibers, melt spun (including melt blown) fibers, synthetic pulp fibers and mixtures thereof. Synthetic fibers may, for example, be comprised of cellulose (often referred to as “rayon”); cellulose derivatives such as esters, ether, or nitrous derivatives; polyolefins (including polyethylene and polypropylene); polyesters (including polyethylene terephthalate); polyamides (often referred to as “nylon”); acrylics; non-cellulosic polymeric carbohydrates (such as starch, chitin and chitin derivatives such as chitosan); and mixtures thereof.

“Fibrous structure” as used herein means a structure that comprises one or more fibers. Nonlimiting examples of processes for making fibrous structures include known wet-laid papermaking processes and air-laid papermaking processes. Such processes typically include steps of preparing a fiber composition, oftentimes referred to as a fiber slurry in wet-laid processes, either wet or dry, and then depositing a plurality of fibers onto a forming wire or belt such that an embryonic fibrous structure is formed, drying and/or bonding the fibers together such that a fibrous structure is formed, and/or further processing the fibrous structure such that a finished fibrous structure is formed. For example, in typical papermaking processes, the finished fibrous structure is the fibrous structure that is wound on the reel at the end of papermaking, but before converting thereof into a sanitary tissue product.

Nonlimiting types of fibrous structures according to the present invention include conventionally felt-pressed fibrous structures; pattern densified fibrous structures; and high-bulk, uncompacted fibrous structures. The fibrous structures may be of a homogeneous or multilayered (two or three or more layers) construction; and the sanitary tissue products made therefrom may be of a single-ply or multi-ply construction.

The fibrous structures may be post-processed, such as by embossing and/or calendaring and/or folding and/or printing images thereon. The fibrous structures may be through-air-dried fibrous structures or conventionally dried fibrous structures. The fibrous structures may be creped or uncreped.

“Sanitary tissue product” comprises one or more fibrous structures, converted or not, that is useful as a wiping implement for post-urinary and post-bowel movement cleaning (toilet tissue), for otorhinolaryngological discharges (facial tissue and/or disposable handkerchiefs), and multi-functional absorbent and cleaning uses (absorbent towels and/or wipes).

“Ply” or “Plies” as used herein means an individual finished fibrous structure optionally to be disposed in a substantially contiguous, face-to-face relationship with other plies, forming a multiple ply finished fibrous structure product and/or sanitary tissue product. It is also contemplated that a single fibrous structure can effectively form two “plies” or multiple “plies”, for example, by being folded on itself.

“Layered” as used herein means that a fibrous structure comprises two or more layers of different fiber compositions (long, short, hardwood, softwood, curled/kinked, linear). Layered fibrous structures are well known in the art as exemplified in U.S. Pat. Nos. 3,994,771, 4,300,981 and 4,166,001 and European Patent Publication No. 613 979 A1. Fibers typically being relatively long softwood and relatively short hardwood fibers are used in multi-layered fibrous structure papermaking processes. Multi-layered fibrous structures suitable for the present invention may comprise at least two superposed layers, an inner layer and at least one outer layer contiguous with the inner layer. Preferably, the multi-layered fibrous structures comprise three superposed layers, an inner or center layer, and two outer layers, with the inner layer located between the two outer layers. The two outer layers preferably comprise a primary filamentary constituent of about 60% or more by weight of relatively short papermaking fibers having an average fiber length, L, of less than about 1.5 mm. These short papermaking fibers are typically hardwood fibers, preferably hardwood Kraft fibers, especially Acacia pulp fibers alone or in combination with other hardwood pulp fibers such as Eucalyptus pulp fibers. The inner layer preferably comprises a primary filamentary constituent of about 60% or more by weight of relatively long papermaking fibers having an average fiber length, L, of greater than or equal to about 1.5 mm. These long papermaking fibers are typically softwood fibers, preferably, northern softwood Kraft fibers.

The fiber compositions forming the layers of the fibrous structure may comprise any mixture of fiber types. The fibrous structures of the present invention may comprise at least two and/or at least three and/or at least four and/or at least five layers.

“Surface of a fibrous structure” as used herein means that portion of the fibrous structure that is exposed to the external environment. In other words, the surface of a fibrous structure is that portion of the fibrous structure that is not completely surrounded by other portions of the fibrous structure.

“User Contacting Surface” as used herein means that portion of the fibrous structure and/or surface treating composition and/or lotion composition present directly and/or indirectly on the surface of the fibrous structure that is exposed to the external environment. In other words, it is that surface formed by the fibrous structure including any surface treating composition and/or lotion composition present directly and/or indirectly on the surface of the fibrous structure that contacts an opposing surface, such as a user's skin, when used by a user. For example, it is that surface formed by the fibrous structure including any surface treating composition and/or lotion composition present directly and/or indirectly on the surface of the fibrous structure that contacts a user's skin when a user wipes his/her skin with the fibrous structure of the present invention.

In one example, the user contacting surface, especially for a textured and/or structured fibrous structure, such as a through-air-dried fibrous structure and/or an embossed fibrous structure, may comprise raised areas and recessed areas of the fibrous structure. In the case of a through-air-dried, pattern densified fibrous structure the raised areas may be knuckles and the recessed areas may be pillows and vice versa. Accordingly, the knuckles may, directly and/or indirectly, comprise the surface treating composition and/or lotion composition and the pillows may be void of the surface treating composition and/or the lotion composition and vice versa. A similar case is true for embossed fibrous structures where the embossed areas may, directly and/or indirectly, comprise the surface treating composition and/or the lotion composition and the non-embossed areas may be void of the surface treating composition and/or the lotion composition and vice versa.

The user contacting surface may be present on the fibrous structure and/or sanitary tissue product before use by the user and/or the user contacting surface may be created/formed prior to and/or during use of the fibrous structure and/or sanitary tissue product by the user, such as upon the user applying pressure to the fibrous structure and/or sanitary tissue product as the user contacts the user's skin with the fibrous structure and/or sanitary tissue product.

All percentages and ratios are calculated by weight unless otherwise indicated. All percentages and ratios are calculated based on the total composition unless otherwise indicated.

Unless otherwise noted, all component or composition levels are in reference to the active level of that component or composition, and are exclusive of impurities, for example, residual solvents or by-products, which may be present in commercially available sources.

### Lotion Composition

The lotion composition of the present invention comprises a microcrystalline wax. In one example, the microcrystalline wax exhibits a melting point of less 85° C. or less and/or 80° or less and/or 76° C. or less and/or 35° C. or greater and/or 40° C. or greater and/or 45° C. or greater. In another example, the microcrystalline wax exhibits a melting point of from about 35° C. to about 85° C. and/or from about 40° C. to about 80° C. and/or from about 45° C. to about 76° C.

In another example, the microcrystalline wax may exhibit a viscosity at 100° C. of less than 25 mm²/second (mm²/sec.) and/or less than 20 mm²/sec. and/or less than 18 mm²/sec. and/or to about 12 mm²/sec. and/or to about 14 mm²/sec. In another example, the microcrystalline wax exhibits a viscosity of from about 12 mm²/sec. to about 25 mm²/sec. and/or from about 14 mm²/sec. to about 20 mm²/sec.

The lotion composition of the present invention may comprise from about 1% to about 50% and/or from about 3% to about 50% and/or from about 5% to about 45% and/or from about 10% to about 40% by weight of a microcrystalline wax.

The lotion composition may comprise oils and/or emollients and/or other waxes and/or other immobilizing agents. In one example, the lotion composition comprises from about 10% to about 90% and/or from about 30% to about 90% and/or from about 40% to about 90% and/or from about 40% to about 85% of an oil and/or emollient. In another example, the lotion composition comprises from about 10% to about 50% and/or from about 15% to about 45% and/or from about 20% to about 40% of an immobilizing agent. In another example, the lotion composition comprises from about 0% to about 60% and/or from about 5% to about 50% and/or from about 5% to about 40% of petrolatum.

In one example, the lotion composition comprises a microcrystalline wax and a paraffin wax. When present, the microcrystalline wax and paraffin wax may each be present in the lotion composition at a level of about 1% to about 50% and/or from about 3% to about 50% and/or from about 5% to about 45% and/or from about 10% to about 40% by weight of the lotion composition. In one example, the lotion composition comprises a microcrystalline wax and a paraffin wax at a weight ratio of microcrystalline wax to paraffin wax of greater than 1 and/or greater than 1.3 and/or greater than 1.5 and/or greater than 1.7 and/or to about 4 and/or to about 3.5 and/or to about 3 and/or to about 2.5. In one example, the microcrystalline wax and paraffin wax are present in the lotion composition of the present invention at a weight ratio of about 2. In another example, the microcrystalline wax is present in the lotion composition at a level of from about 5% to about 20% and/or from about 10% to about 15% by weight and the paraffin wax may be present in the lotion composition at a level of from about 1% to about 15% and/or from about 3% to about 10% by weight.

The lotion compositions may be heterogeneous. They may contain solids, gel structures, polymeric material, a multiplicity of phases (such as oily and water phase) and/or emulsified components. It may be difficult to determine precisely the melting temperature of the lotion composition, i.e. difficult to determine the temperature of transition between the liquid form, the quasi-liquid from, the quasi-solid form and the solid form. The terms melting temperature, melting point, transition point and transition temperature are used interchangeably in this document and have the same meaning.

The lotion compositions may be semi-solid, of high viscosity so they do not substantially flow without activation during the life of the product or gel structures.

The lotion compositions may be shear thinning and/or they may strongly change their viscosity around skin temperature to allow for transfer and easy spreading on a user's skin.

The lotion compositions may be in the form of emulsions and/or dispersions. The lotion composition may be a transferable lotion composition.

In one example of a lotion composition, the lotion composition has a water content of less than about 20% and/or less than 10% and/or less than about 5% or less than about 0.5%.

In another example, the lotion composition may have a solids content of at least about 15% and/or at least about 25% and/or at least about 30% and/or at least about 40% to about 100% and/or to about 95% and/or to about 90% and/or to about 80%.

A nonlimiting example of a suitable lotion composition of the present invention comprises a chemical softening agent, such as an emollient, that softens, soothes, supples, coats, lubricates, or moisturizes the skin. The lotion composition may sooth, moisturize, and/or lubricate a user's skin.

The lotion composition may comprise an oil and/or an emollient. Nonlimiting examples of suitable oils and/or emollients include glycols (such as propylene glycol and/or glycerine), polyglycols (such as triethylene glycol), petrolatum, fatty acids, fatty alcohols, fatty alcohol ethoxylates, fatty alcohol esters and fatty alcohol ethers, fatty acid ethoxylates, fatty acid amides and fatty acid esters, hydrocarbon oils (such as mineral oil), squalane, fluorinated emollients, silicone oil (such as dimethicone) and mixtures thereof.

Nonlimiting examples of emollients useful in the present invention can be petroleum-based, fatty acid ester type, alkyl ethoxylate type, or mixtures of these materials. Suitable petroleum-based emollients include those hydrocarbons, or mixtures of hydrocarbons, having chain lengths of from 16 to 32 carbon atoms. Petroleum based hydrocarbons having these chain lengths include petrolatum (also known as “mineral wax,” “petroleum jelly” and “mineral jelly”). Petrolatum usually refers to more viscous mixtures of hydrocarbons having from 16 to 32 carbon atoms. A suitable Petrolatum is available from Witco, Corp., Greenwich, Conn. as White Protopet® 1 S.

Suitable fatty acid ester emollients include those derived from long chain C₁₂-C₂₈ fatty acids, such as C₁₆-C₂₂ saturated fatty acids, and short chain C₁-C₈ monohydric alcohols, such as C₁-C₃ monohydric alcohols. Nonlimiting examples of suitable fatty acid ester emollients include methyl palmitate, methyl stearate, isopropyl laurate, isopropyl myristate, isopropyl palmitate, and ethylhexyl palmitate. Suitable fatty acid ester emollients can also be derived from esters of longer chain fatty alcohols (C₁₂-C₂₈, such as C₁₂-C₁₆) and shorter chain fatty acids e.g., lactic acid, such as lauryl lactate and cetyl lactate.

Suitable fatty acid ester type emollients include those derived from C₁₂-C₂₈ fatty acids, such as C₁₆-C₂₂ saturated fatty acids, and short chain (C₁-C₈ and/or C₁-C₃) monohydric alcohols. Representative examples of such esters include methyl palmitate, methyl stearate, isopropyl laurate, isopropyl myristate, isopropyl palmitate, and ethylhexyl palmitate. Suitable fatty acid ester emollients can also be derived from esters of longer chain fatty alcohols (C₁₂-C₂₈ and/or C₁₂-C₁₆) and shorter chain fatty acids e.g., lactic acid, such as lauryl lactate and cetyl lactate.

Suitable alkyl ethoxylate type emollients include C₁₂-C₁₈ fatty alcohol ethoxylates having an average of from 3 to 30 oxyethylene units, such as from about 4 to about 23. Nonlimiting examples of such alkyl ethoxylates include laureth-3 (a lauryl ethoxylate having an average of 3 oxyethylene units), laureth-23 (a lauryl ethoxylate having an average of 23 oxyethylene units), ceteth-10 (acetyl ethoxylate having an average of 10 oxyethylene units), steareth-2 (a stearyl ethoxylate having an average of 2 oxyethylene units) and steareth-10 (a stearyl ethoxylate having an average of 10 oxyethylene units). These alkyl ethoxylate emollients are typically used in combination with the petroleum-based emollients, such as petrolatum, at a weight ratio of alkyl ethoxylate emollient to petroleum-based emollient of from about 1:1 to about 1:3, for example from about 1:1.5 to about 1:2.5.

The lotion compositions of the present invention may include other “immobilizing agents”, so-called because they are believed to act to prevent migration of the emollient so that it can remain primarily on the surface of the fibrous structure to which it is applied so that it may deliver maximum softening benefit as well as be available for transferability to the user's skin. Suitable immobilizing agents for the present invention can comprise polyhydroxy fatty acid esters, polyhydroxy fatty acid amides, and mixtures thereof. To be useful as immobilizing agents, the polyhydroxy moiety of the ester or amide should have at least two free hydroxy groups. It is believed that these free hydroxy groups are the ones that co-crosslink through hydrogen bonds with the cellulosic fibers of the tissue paper web to which the lotion composition is applied and homo-crosslink, also through hydrogen bonds, the hydroxy groups of the ester or amide, thus entrapping and immobilizing the other components in the lotion matrix. Nonlimiting examples of suitable esters and amides will have three or more free hydroxy groups on the polyhydroxy moiety and are typically nonionic in character. Because of the skin sensitivity of those using paper products to which the lotion composition is applied, these esters and amides should also be relatively mild and non-irritating to the skin.

Suitable polyhydroxy fatty acid esters for use in the present invention will have the formula:

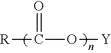

wherein R is a C₅-C₃₁ hydrocarbyl group, such as a straight chain C₇-C₁₉ alkyl or alkenyl and/or a straight chain C₉-C₁₇ alkyl or alkenyl and/or a straight chain C₁₁-C₁₇ alkyl or alkenyl, or mixture thereof; Y is a polyhydroxyhydrocarbyl moiety having a hydrocarbyl chain with at least 2 free hydroxyls directly connected to the chain; and n is at least 1. Suitable Y groups can be derived from polyols such as glycerol, pentaerythritol; sugars such as raffinose, maltodextrose, galactose, sucrose, glucose, xylose, fructose, maltose, lactose, mannose and erythrose; sugar alcohols such as erythritol, xylitol, malitol, mannitol and sorbitol; and anhydrides of sugar alcohols such as sorbitan.

One class of suitable polyhydroxy fatty acid esters for use in the present invention comprises certain sorbitan esters, such as sorbitan esters of C₁₆-C₂₂ saturated fatty acids.

Immobilizing agents include agents that are may prevent migration of the emollient into the fibrous structure such that the emollient remain primarily on the surface of the fibrous structure and/or sanitary tissue product and/or on the surface treating composition on a surface of the fibrous structure and/or sanitary tissue product and facilitate transfer of the lotion composition to a user's skin. Immobilizing agents may function as viscosity increasing agents and/or gelling agents.

Nonlimiting examples of suitable immobilizing agents include waxes (such as ceresin wax, ozokerite, other microcrystalline waxes, petroleum waxes, fisher tropsh waxes, silicone waxes, paraffin waxes), fatty alcohols (such as cetyl, cetaryl, cetearyl and/or stearyl alcohol), fatty acids and their salts (such as metal salts of stearic acid), mono and polyhydroxy fatty acid esters, mono and polyhydroxy fatty acid amides, silica and silica derivatives, gelling agents, thickeners and mixtures thereof.

In one example, the lotion composition comprises at least one immobilizing agent and at least one emollient.

In another example, the lotion composition may comprise one or more volatile agents such as menthol (such as L-menthol), camphor, eucalyptus oil, lavender oil (such as Bulgarian Lavender Oil) and mixtures thereof.

In one example, the lotion composition comprises a skin benefit agent.

In another example, the lotion composition of the present invention comprises less than 5% and/or less than 3% and/or less than 1% and/or less than 0.5% and/or 0% by weight of an alkyl ethoxylate type emollient, for example a C₁₂-C₁₈ fatty alcohol ethoxylate having an average of from 3 to 30 oxyethylene units, such as steareth-2 (a stearyl ethoxylate having an average of 2 oxyethylene units) and/or steareth-10 (a stearyl ethoxylate having an average of 10 oxyethylene units).

### Skin Benefit Agent

One or more skin benefit agents may be included in the lotion composition of the present invention. If a skin benefit agent is included in the lotion composition, it may be present in the lotion composition at a level of from about 0.5% to about 80% and/or 0.5% to about 70% and/or from about 5% to about 60% by weight of the lotion.

Nonlimiting examples of skin benefit agents include zinc oxide, vitamins, such as Vitamin B3 and/or Vitamin E, sucrose esters of fatty acids, such as Sefose 1618S (commercially available from Procter & Gamble Chemicals), antiviral agents, anti-inflammatory compounds, lipid, inorganic anions, inorganic cations, protease inhibitors, sequestration agents, chamomile extracts, aloe vera, calendula officinalis, alpha bisalbolol, Vitamin E acetate and mixtures thereof.

Nonlimiting examples of suitable skin benefit agents include fats, fatty acids, fatty acid esters, fatty alcohols, triglycerides, phospholipids, mineral oils, essential oils, sterols, sterol esters, emollients, waxes, humectants and combinations thereof.

### Other Ingredients in Lotion Composition

Other optional ingredients that may be included in the lotion composition include vehicles, shea butter, perfumes, especially long lasting and/or enduring perfumes, antibacterial actives, antiviral actives, disinfectants, pharmaceutical actives, film formers, deodorants, opacifiers, astringents and solvents.

### Vehicle

As used herein a “vehicle” is a material that can be used to dilute and/or emulsify agents forming the surface treating composition and/or lotion composition to form a dispersion/emulsion. Suitable materials for use as the vehicle of the present invention include hydroxyl functional liquids, including but not limited to water.

### Process Aids

Process aids may also be used in the lotion compositions of the present invention. Nonlimiting examples of suitable process aids include brighteners, such as TINOPAL CBS-X®, obtainable from CIBA-GEIGY of Greensboro, N.C.

### Fibrous Structure

The lotion composition of the present invention may be applied to a fibrous structure. The fibrous structure of the present invention comprises a lotion composition of the present invention and may further comprise a surface softening composition. When the fibrous structure comprises a lotion composition and a surface softening composition, the surface softening composition may be sandwiched between the lotion composition, which forms a user contacting surface, and the surface of the fibrous structure. The lotion composition and the surface softening composition may be phase registered such that the user contacting surface comprises lotion composition regions and fibrous structure regions.

When the fibrous structure comprises a lotion composition without a surface softening, the lotion composition is in contact with the surface of the fibrous structure, thus creating a user contacting surface comprising the lotion composition. The user contacting surface may be comprised entirely of the lotion composition or it may be comprised of regions of lotion composition and regions of fibrous structure.

The fibrous structure and/or sanitary tissue product of the present invention may comprise greater than 0.5 and/or greater than 1 and/or greater than 1.5 and/or greater than 2 and/or less than 10 and/or less than 8 and/or less than 7 and/or less than 6 lbs/3000 ft² of the lotion composition.

In one example, the fibrous structure and/or sanitary tissue product of the present invention may comprise greater than 0.5 and/or greater than 1 and/or greater than 1.5 and/or greater than 2 and/or less than 10 and/or less than 8 and/or less than 7 and/or less than 6 lbs/3000 ft² of the lotion composition and optionally, any surface treating composition.

In another example, the fibrous structure and/or sanitary tissue product may comprise from about 0.01% to about 20% and/or from about 0.05% to about 15% and/or from about 0.1% to about 10% and/or from about 0.01% to about 5% and/or from about 0.1% to about 2% of total basis weight of the lotion composition and optionally, any surface treating composition.

Optional ingredients may also be present in and/or on the fibrous structure. Such optional ingredients may include essential oils and other ingredients such as cedar leaf oil, nutmeg oil, turpentine oil, thymol, wet strength agents, dry strength agents, antiviral agents, including organic acids, perfumes, especially long lasting and/or enduring perfumes, antibacterial agents, opacifiers, wetting agents, lint resisting agents, absorbency-enhancing agents, polyol polyesters, antimigration agents, polyhydroxy plasticizers and mixtures thereof. Such optional ingredients may be added to the fiber furnish, the embryonic fibrous web and/or the fibrous structure.

Such optional ingredients may be present in the fibrous structures at any level based on the dry weight of the fibrous structure. The optional ingredients may be present in the fibrous structures at a level of from about 0.001 to about 50% and/or from about 0.001 to about 20% and/or from about 0.01 to about 5% and/or from about 0.03 to about 3% and/or from about 0.1 to about 1.0% by weight, on a dry fibrous structure basis.

### Surface Treating Composition

A surface treating composition, for purposes of the present invention, is a composition that improves the tactile sensation of a surface of a fibrous structure perceived by a user whom holds a fibrous structure and/or sanitary tissue product comprising the fibrous structure and rubs it across the user's skin. Such tactile perceivable softness can be characterized by, but is not limited to, friction, flexibility, and smoothness, as well as subjective descriptors, such as a feeling like lubricious, velvet, silk or flannel.

The surface treating composition may or may not be transferable. Typically, it is substantially non-transferable.

The surface treating composition may increase or decrease the surface friction of the surface of the fibrous structure, especially the user contacting surface of the fibrous structure. Typically, the surface treating composition will reduce the surface friction of the surface of the fibrous structure compared to a surface of the fibrous structure without such surface treating composition.

The surface treating composition may have a wettability tension less than or equal to the surface tension of a lotion composition applied to a surface of a fibrous structure treated with the surface treating composition so as to minimize the spreading of the lotion composition that comes into contact with the surface treating composition and/or to reduce and/or inhibit migration of the lotion composition into the fibrous structure.

The surface treating composition comprises a surface treating agent. The surface treating composition during application to the fibrous structure may comprise at least about 0.1% and/or at least 0.5% and/or at least about 1% and/or at least about 3% and/or at least about 5% to about 90% and/or to about 80% and/or to about 70% and/or to about 50% and/or to about 40% by weight of the surface treating agent. In one example, the surface treating composition comprises from about 5% to about 40% by weight of the surface treating agent.

The surface treating composition present on the fibrous structure and/or sanitary tissue product comprising the fibrous structure of the present invention may comprise at least about 0.01% and/or at least about 0.05% and/or at least about 0.1% of total basis weight of the surface treating agent. In one example, the fibrous structure and/or sanitary tissue product may comprise from about 0.01% to about 20% and/or from about 0.05% to about 15% and/or from about 0.1% to about 10% and/or from about 0.01% to about 5% and/or from about 0.1% to about 2% of total basis weight of the surface treating composition.

Nonlimiting examples of suitable surface treating agents can be selected from the group consisting of: polymers such as polyethylene and derivatives thereof, hydrocarbons, waxes, oils, silicones, organosilicones (oil compatible), quaternary ammonium compounds, fluorocarbons, substituted C₁₀-C₂₂ alkanes, substituted C₁₀-C₂₂ alkenes, in particular derivatives of fatty alcohols and fatty acids (such as fatty acid amides, fatty acid condensates and fatty alcohol condensates), polyols, derivatives of polyols (such as esters and ethers), sugar derivatives (such as ethers and esters), polyglycols (such as polyethyleneglycol) and mixtures thereof.

In one example, the surface treating composition of the present invention is a microemulsion and/or a macroemulsion of a surface treating agent (for example an aminofunctional polydimethylsiloxane, specifically an aminoethylaminopropyl polydimethylsiloxane) in water. In such an example, the concentration of the surface treating agent within the surface treating composition may be from about 3% to about 60% and/or from about 4% to about 50% and/or from about 5% to about 40%. A nonlimiting examples of such microemulsions are commercially available from Wacker Chemie (MR1003, MR103, MR102). A nonlimiting example of such a macroemulsion is commercially available from General Electric Silicones (CM849).

Nonlimiting examples of suitable waxes may be selected from the group consisting of: paraffin, polyethylene waxes, beeswax and mixtures thereof.

Nonlimiting examples of suitable oils may be selected from the group consisting of: mineral oil, silicone oil, silicone gels, petrolatum and mixtures thereof.

Nonlimiting examples of suitable silicones may be selected from the group consisting of: polydimethylsiloxanes, aminosilicones, cationic silicones, quaternary silicones, silicone betaines and mixtures thereof.

Nonlimiting examples of suitable polysiloxanes and/or monomeric/oligomeric units may be selected from the compounds having monomeric siloxane units of the following structure:

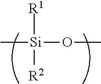

wherein, R¹ and R2, for each independent siloxane monomeric unit can each independently be hydrogen or any alkyl, aryl, alkenyl, alkaryl, aralkyl, cycloalkyl, halogenated hydrocarbon, or other radical. Any of such radical can be substituted or unsubstituted. R¹ and R² radicals of any particular monomeric unit may differ from the corresponding functionalities of the next adjoining monomeric unit. Additionally, the polysiloxane can be either a straight chain, a branched chain or have a cyclic structure. The radicals R¹ and R² can additionally independently be other silaceous functionalities such as, but not limited to siloxanes, polysiloxanes, silanes, and polysilanes. The radicals R¹ and R² may contain any of a variety of organic functionalities including, for example, alcohol, carboxylic acid, phenyl, and amine functionalities. The end groups can be reactive (alkoxy or hydroxyl) or nonreactive (trimethylsiloxy). The polymer can be branched or unbranched.

In one example, suitable polysiloxanes include straight chain organopolysiloxane materials of the following general formula:

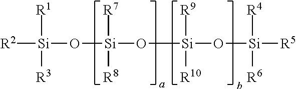

wherein each R¹-R⁹ radical can independently be any C₁-C₁₀ unsubstituted alkyl or aryl radical, and R¹⁰ of any substituted C₁-C₁₀ alkyl or aryl radical. In one example, each R¹-R⁹ radical is independently any C₁-C₄ unsubstituted alkyl group. Those skilled in the art will recognize that technically there is no difference whether, for example, R⁹ or R¹⁰ is the substituted radical. In another example, the mole ratio of b to (a+b) is between 0 and about 20% and/or between 0 and about 10% and/or between about 1% and about 5%.

A nonlimiting example of a cationic silicone polymer that can be used as a surface treating agent comprises one or more polysiloxane units, preferably polydimethylsiloxane units of formula —{(CH₃)₂SiO}_{c}— having a degree of polymerization, c, of from about 1 to about 1000 and/or from about 20 to about 500 and/or from about 50 to about 300 and/or from about 100 to about 200, and organosilicone-free units comprising at least one diquaternary unit. In one example, the cationic silicone polymer has from about 0.05 to about 1.0 and/or from about 0.2 to about 0.95 and/or from about 0.5 to about 0.9 mole fraction of the organosilicone-free units selected from cationic divalent organic moieties. The cationic divalent organic moiety may be selected from N,N,N′,N′-tetramethyl-1,6-hexanediammonium units.

The cationic silicone polymer may contain from about 0 to about 0.95 and/or from about 0.001 to about 0.5 and/or from about 0.05 to about 0.2 mole fraction of the total of organosilicone-free units, polyalkyleneoxide amines of the following formula:

[—Y—O(—CₐH₂ₐO)_{b}—Y—]

wherein Y is a divalent organic group comprising a secondary or tertiary amine, such as a C₁ to C₈ alkyleneamine residue; a is from 2 to 4, and b is from 0 to 100.

Such polyalkyleneoxide amine—containing units can be obtained by introducing in the silicone polymer structure, compounds such as those sold under the tradename Jeffamine® from Huntsman Corporation. A preferred Jeffamine is Jeffamine ED-2003.

The cationic silicone polymer may contain from about 0 and/or from about 0.001 to about 0.2 mole fraction, of the total of organosilicone-free units, of —NR₃+ wherein R is alkyl, hydroxyalkyl or phenyl. These units can be thought of as end-caps.

Moreover the cationic silicone polymer generally contains anions, selected from inorganic and organic anions.

A nonlimiting example of a cationic silicone polymer comprises one or more polysiloxane units and one or more quaternary nitrogen moieties, and includes polymers wherein the cationic silicone polymer has the formula:

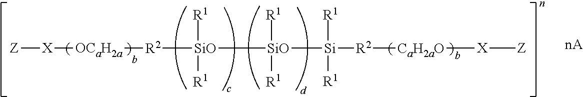

wherein:

R¹ is independently selected from the group consisting of: C₁₋₂₂ alkyl, C₂₋₂₂ alkenyl, C₆₋₂₂ alkylaryl, aryl, cycloalkyl, and mixtures thereof;

R² is independently selected from the group consisting of: divalent organic moieties that may contain one or more oxygen atoms (such moieties preferably consist essentially of C and H or of C, H and O);

X is independently selected from the group consisting of ring-opened epoxides;

R³ is independently selected from polyether groups having the formula:

-M¹(CₐH₂ₐO)_{b}-M²

wherein M¹ is a divalent hydrocarbon residue; M² is independently selected from the group consisting of H, C₁₋₂₂ alkyl, C₂₋₂₂ alkenyl, C₆₋₂₂ alkylaryl, aryl, cycloalkyl, C₁₋₂₂ hydroxyalkyl, polyalkyleneoxide, (poly)alkoxy alkyl, and mixtures thereof;

Z is independently selected from the group consisting of monovalent organic moieties comprising at least one quaternized nitrogen atom;

a is from 2 to 4; b is from 0 to 100; c is from 1 to 1000 and/or greater than 20 and/or greater than 50 and/or less than 500 and/or less than 300 and/or from 100 to 200;

d is from 0 to 100; n is the number of positive charges associated with the cationic silicone polymer, which is greater than or equal to 2; and A is a monovalent anion.

Another nonlimiting example of a cationic silicone polymer comprises one or more polysiloxane units and one or more quaternary nitrogen moieties, and includes polymers wherein the cationic silicone polymer has the formula:

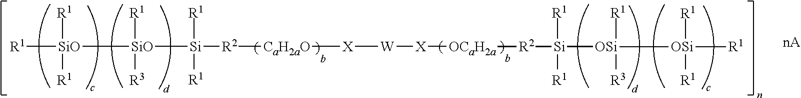

wherein:

R¹ is independently selected from the group consisting of: C₁₋₂₂ alkyl, C₂₋₂₂ alkenyl, C₆₋₂₂ alkylaryl, aryl, cycloalkyl, and mixtures thereof;

R² is independently selected from the group consisting of: divalent organic moieties that may contain one or more oxygen atoms;

X is independently selected from the group consisting of ring-opened epoxides;

R³ is independently selected from polyether groups having the formula:

-M(CₐH₂ₐO)_{b}-M²

wherein M¹ is a divalent hydrocarbon residue; M² is independently selected from the group consisting of H, C₁₋₂₂ alkyl, C₂₋₂₂ alkenyl, C₆₋₂₂ alkylaryl, aryl, cycloalkyl, C₁₋₂₂ hydroxyalkyl, polyalkyleneoxide, (poly)alkoxy alkyl, and mixtures thereof;

X is independently selected from the group consisting of ring-opened epoxides;

W is independently selected from the group consisting of divalent organic moieties comprising at least one quaternized nitrogen atom;

a is from 2 to 4; b is from 0 to 100; c is from 1 to 1000 and/or greater than 20 and/or greater than 50 and/or less than 500 and/or less than 300 and/or from 100 to 200; d is from 0 to 100; n is the number of positive charges associated with the cationic silicone polymer, which is greater than or equal to 1; and A is a monovalent anion, in other words, a suitable counterion.

References disclosing nonlimiting examples of suitable polysiloxanes include U.S. Pat. Nos. 2,826,551, 3,964,500, 4,364,837, 5,059,282, 5,529,665, 5,552,020 and British Patent No. 849,433 and Silicone Compounds, pp. 181-217, distributed by Petrach Systems, Inc., which contains an extensive listing and description of polysiloxanes in general.

Viscosity of polysiloxanes useful for this invention may vary as widely as the viscosity of polysiloxanes in general vary, so long as the polysiloxane can be rendered into a form which can be applied to the fibrous structures herein. This includes, but is not limited to, viscosity as low as about 25 centistokes to about 20,000,000 centistokes or even higher.

Nonlimiting examples of suitable quaternary ammonium compounds may be selected from compounds having the formula:

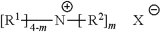

wherein:
m is 1 to 3; each R¹ is independently a C₁-C₆ alkyl group, hydroxyalkyl group, hydrocarbyl or substituted hydrocarbyl group, alkoxylated group, benzyl group, or mixtures thereof; each R² is independently a C₁₄-C₂₂ alkyl group, hydroxyalkyl group, hydrocarbyl or substituted hydrocarbyl group, alkoxylated group, benzyl group, or mixtures thereof; and X⁻ is any quaternary ammonium-compatible anion.

In another example, the quaternary ammonium compounds may be mono or diester variations having the formula:

(R¹)₄₋ₘ—N+—[(CH₂)ₙ—Y—R³]ₘX⁻

wherein:
Y is —O—(O)C—, or —C(O)—O—, or —NH C(O)—, or —C(O)—NH—; m is 1 to 3; n is 0 to 4; each R¹ is independently a C₁-C₆ alkyl group, hydroxyalkyl group, hydrocarbyl or substituted hydrocarbyl group, alkoxylated group, benzyl group, or mixtures thereof; each R³ is independently a C₁₃-C₂₁ alkyl group, hydroxyalkyl group, hydrocarbyl or substituted hydrocarbyl group, alkoxylated group, benzyl group, or mixtures thereof, and X⁻ is any quaternary ammonium-compatible anion.

In another example, the quaternary ammonium compound may be an imidazolinium compound, such as an imidazolinium salt.

As mentioned above, X⁻ can be any quaternary ammonium-compatible anion, for example, acetate, chloride, bromide, methyl sulfate, formate, sulfate, nitrate and the like can also be used in the present invention. In one example, X⁻ is chloride or methyl sulfate.

The surface treating composition may comprise additional ingredients such as a vehicle as described herein below which may not be present on the fibrous structure and/or sanitary tissue product comprising such fibrous structure. In one example, the surface treating composition may comprise a surface treating agent and a vehicle such as water to facilitate the application of the surface treating agent onto the surface of the fibrous structure.

Nonlimiting examples of quaternary ammonium compounds suitable for use in the present invention include the well-known dialkyldimethylammonium salts such as ditallowedimethylammonium chloride, ditallowedimethylammonium methylsulfate, di(hydrogenated tallow)dimethylammonium chloride. In one example, the surface treating composition comprises di(hydrogenated tallow)dimethylammonium chloride, commercially available from Witco Chemical Company Inc. of Dublin, Ohio as Varisoft 137®.

Nonlimiting examples of ester-functional quaternary ammonium compounds having the structures named above and suitable for use in the present invention include the well-known diester dialkyl dimethyl ammonium salts such as diester ditallow dimethyl ammonium chloride, monoester ditallow dimethyl ammonium chloride, diester ditallow dimethyl ammonium methyl sulfate, diester di(hydrogenated)tallow dimethyl ammonium methyl sulfate, diester di(hydrogenated)tallow dimethyl ammonium chloride, and mixtures thereof. In one example, the surface treating composition comprises diester ditallow dimethyl ammonium chloride and/or diester di(hydrogenated)tallow dimethyl ammonium chloride, both commercially available from Witco Chemical Company Inc. of Dublin, Ohio under the tradename “ADOGEN SDMC”.

In one example, the surface treating composition comprises a “polyhydroxy compound”, which as used herein is a chemical agent that imparts lubricity or emolliency to tissue paper products and also possesses permanence with regard to maintaining the fidelity of its deposits without substantial migration when exposed to the environmental conditions to which products of this type are ordinarily exposed during their typical life cycle. The present invention contains as an essential component from about 2.0% to about 30.0%, preferably from 5% to about 20.0%, more preferably from about 8.0% to about 15.0%, of a water soluble polyhydroxy compound based on the dry fiber weight of the tissue paper. In another embodiment, the present invention may contain as an essential component an application of from about 0.1 g/m² to about 36 g/m², preferably from about 0.55 g/m² to about 20 g/m² more preferably from about 0.65 g/m² to about 12 g/m², of a water soluble polyhydroxy compound to the tissue paper.

Examples of water soluble polyhydroxy compounds suitable for use in the present invention include glycerol, polyglycerols having a weight average molecular weight of from about 150 to about 800 and polyoxyethylene and polyoxypropylene having a weight-average molecular weight of from about 200 to about 4000, preferably from about 200 to about 1000, most preferably from about 200 to about 600. Polyoxyethylene having a weight average molecular weight of from about 200 to about 600 are especially preferred. Mixtures of the above-described polyhydroxy compounds may also be used. For example, mixtures of glycerol and polyglycerols, mixtures of glycerol and polyoxyethylenes, ‘mixtures of polyglycerols and polyoxyethylenes, etc. are useful in the present invention. A particularly preferred polyhydroxy compound is polyoxyethylene having a weight average molecular weight of about 200. This material is available commercially from the BASF Corporation of Florham Park, N.J. under the trade names “Pluriol E200” and “Pluracol E200”.

### Method of Making Lotion Composition

An example of a method for making a lotion composition of the present invention comprises the step of combining a microcrystalline wax with an oil to form the lotion composition.

### Properties of Lotion Composition

The lotion composition of the present invention provides novel properties as set forth in Table 1 below.

| | Microcrystalline | | Viscosity | Viscosity @ | Kinetic |
|---|---|---|---|---|---|
| | Wax | Penetration | @ 60° C. | 65° C. | Coefficient of |
| Sample | (Y/N) | Hardness | (1/s) | (1/s) | Friction |
| Puffs Plus ® | N | 62 | — | 7 | 1.104 |
| Lotion¹ | | | | | |
| Puffs Plus ® | N | 52 | — | 18 | 1.547 |
| Lotion² | | | | | |
| U.S. Pat. No. | Y | — | 77 | — | — |
| 7,485,373 | | | | | |
| Lotion | | | | | |
| Composition | | | | | |
| Table 4 E | | | | | |
| U.S. Pat. No. | Y | — | 58 | — | — |
| 7,485,373 | | | | | |
| Lotion | | | | | |
| Composition | | | | | |
| Table 4 F | | | | | |
| Lotion | Y | 46 | | 46 | 0.664 |
| Composition of | | | | | |
| Present | | | | | |
| Invention | | | | | |

¹Lotion composition present on pre-2011 commercially available Puffs Plus ® Lotion product.
²Lotion composition present on 2011 commercially available Puffs Plus ® Lotion product.

Further, the lotion composition provides the fibrous structure and/or sanitary tissue product of the present invention with improved softness, such as determined/measured using a spot/feel panel, for example a spot/feel expert panel, compared to lotion compositions void of microcrystalline wax.

### Article of Manufacture

In one example, an article of manufacture comprising a carton defining an interior volume and one or more fibrous structures of the present invention removably housed in the interior volume of the carton is provided.

The carton may be a paperboard carton, for example a CCNB board carton.

The carton may comprise a coating, such as a protective coating that prevents migration of lotion compositions into and/or through the carton. A non-limiting example of a carton that comprises a coating is MillMask® commercially available from RockTerm Converting Company. In one example, the carton may be void of such a coating.

In one example, the lotion composition of the present invention exhibits a Lotion Diffusion into Non-Coated Carton of less than 0.4 g of lotion at 5 days at 120° F. as measured according to the Lotion Diffusion into Non-Coated Carton Test Method.

In another example, the lotion composition of the present invention exhibits a Lotion Diffusion into Non-Coated Carton of less than 0.7 at 20 days at 120° F. as measured according to the Lotion Diffusion into Non-Coated Carton Test Method.

Table 2 below sets for the data for the Lotion Diffusion into Non-Coated Carton.

TABLE 2

| | | Lotion | Lotion | Lotion |
|---|---|---|---|---|
| | Lotion Amt (g) | Amt (g) | Amt (g) | Amt (g) |
| | Diffused into | Diffused into | Diffused into | Diffused into |
| | Non-Coated | Non-Coated | Non-Coated | Non-Coated |
| | Carton @ | Carton @ 10 | Carton @ 15 | Carton @ 20 |
| Sample | 5 days | days | days | days |
| Puffs Plus ® | ~0.18 | ~0.6 | ~0.7 | ~0.75 |
| Lotion¹ | | | | |
| Fibrous | ~0.11 | ~0.12 | ~0.13 | ~0.14 |
| Structure | | | | |
| with Lotion | | | | |
| Composition | | | | |
| of Present | | | | |
| Invention | | | | |

¹2011 Commercially available Puffs Plus ® Lotion product.

### Non-Limiting Examples

Lotion compositions according to the present invention are shown below in Table 3.

TABLE 3

| | Example 1 | Example 2 |
|---|---|---|
| Ingredient | Wt % | Wt % |
| Mineral Oil | 55 | 55 |
| Cetearyl alcohol | 18 | 26 |
| Paraffin Wax | 5 | 6 |
| Microcrystalline Wax | 9 | 12 |
| Steareth-2 | 11 | 0 |
| Optional Ingredients (i.e., | Balance | Balance |
| aloe, shea butter, vitamins) | | |

### Test Methods

Unless otherwise indicated, all tests described herein including those described under the Definitions section and the following test methods are conducted on samples that have been conditioned in a conditioned room at a temperature of 73° F.±4° F. (about 23° C.±2.2° C.) and a relative humidity of 50%±10% for 2 hours prior to the test unless otherwise indicated. Samples conditioned as described herein are considered dry samples for purposes of this invention. Further, all tests are conducted in such conditioned room.

### Penetration Hardness Test Method

Penetration Hardness means needle penetration as measured according to ASTM D 1321 Needle Penetration of Petroleum Waxes.

### Kinetic Coefficient of Friction Test Method

The Kinetic Coefficient of Friction of a sample is measured according to the following procedure.
Heat the lotion on the hot plate
Make a bad out of Alumina foil with edges, place 4 by 8 inches plate inside the bad.
Pour the hot melt lotion into the bad, forming a thin film of lotion
Let the lotion cool down and solidify
Take the plate out of the alumina foil by cutting the edges
Take the lotion thin cast to the physical test lab to the Davids Loebker Cof of Friction Instrument
Clamp the Lotion Plate from one side to make sure it will not move
Place collagen to cover 20 g (3.5 by 3.5) square sled (Ask for David Loebker for collagen)
Place the sled on default position
Place the sled on the lotion film
Slide the sled over the lotion film for 20 seconds
Collect the results

The dimensions and values disclosed herein are not to be understood as being strictly limited to the exact numerical values recited. Instead, unless otherwise specified, each such dimension is intended to mean both the recited value and a functionally equivalent range surrounding that value. For example, a dimension disclosed as “40 mm” is intended to mean “about 40 mm.”

Every document cited herein, including any cross referenced or related patent or application, is hereby incorporated herein by reference in its entirety unless expressly excluded or otherwise limited. The citation of any document is not an admission that it is prior art with respect to any invention disclosed or claimed herein or that it alone, or in any combination with any other reference or references, teaches, suggests or discloses any such invention. Further, to the extent that any meaning or definition of a term in this document conflicts with any meaning or definition of the same term in a document incorporated by reference, the meaning or definition assigned to that term in this document shall govern.

While particular embodiments of the present invention have been illustrated and described, it would be obvious to those skilled in the art that various other changes and modifications can be made without departing from the spirit and scope of the invention. It is therefore intended to cover in the appended claims all such changes and modifications that are within the scope of this invention.

What is claimed is:

1. A lotion composition comprising a microcrystalline wax that exhibits a Penetration Hardness of 65 or less as measured according to the Penetration Hardness Test Method described herein.

2. The lotion composition according to claim 1 wherein the microcrystalline wax exhibits a melting point of 85° C. or less.

3. The lotion composition according to claim 1 wherein the microcrystalline wax exhibits a viscosity at 100° C. of less than 25 mm²/sec.

4. The lotion composition according to claim 1 wherein the lotion composition further comprises a paraffin wax.

5. The lotion composition according to claim 4 wherein the microcrystalline wax and paraffin wax are present in the lotion composition at a weight ratio of microcrystalline wax to paraffin wax of less than 4.

6. The lotion composition according to claim 1 wherein the lotion composition further comprises a vitamin.

7. The lotion composition according to claim 1 wherein the lotion composition further comprises aloe.

8. The lotion composition according to claim 1 wherein the lotion composition further comprises shea butter.

9. The lotion composition according to claim 1 wherein the lotion composition further comprises an oil.

10. The lotion composition according to claim 9 wherein the oil comprises mineral oil.

11. The lotion composition according to claim 1 wherein the lotion composition exhibits a viscosity at 65° C. of greater than 15 (l/second).

12. The lotion composition according to claim 1 wherein the lotion composition exhibits a Kinetic Coefficient of Friction of less than 1.

13. The lotion composition according to claim 1 wherein lotion composition exhibits a Lotion Diffusion into a Non-Coated Carton of less than 0.4 g of lotion at 5 days at 120° F. as measured according to the Lotion Diffusion into Non-Coated Carton Test Method.

14. The lotion composition according to claim 1 wherein the lotion composition exhibits a Lotion Diffusion into a Non-Coated Carton of less than 0.7 at 20 days at 120° F. as measured according to the Lotion Diffusion into a Non-Coated Carton Test Method.

15. A method for making a lotion composition, the method comprising the step of combining a microcrystalline wax that exhibits a Penetration Hardness of 65 or less as measured according to the Penetration Hardness Test Method described herein with an oil to form the lotion composition.

16. A fibrous structure comprising a surface, the surface comprising a lotion composition according to claim 1.

17. The fibrous structure according to claim 16 wherein the fibrous structure comprises at least 1% by weight of the lotion composition.

18. The fibrous structure according to claim 16 wherein the fibrous structure further comprises a surface treating composition.

19. The fibrous structure according to claim 18 wherein the surface treating composition comprises a polyhydroxy compound.

20. An article of manufacture comprising a carton defining an interior volume and one or more fibrous structures according to claim 16 removably housed in the interior volume of the carton.
